(19) United States
(12) Patent Application Publication
(10) Pub. No.: US 2024/0369557 A1
(43) Pub. Date: Nov. 7, 2024

(54) A BLOOD PROFILE ANALYSIS METHOD FOR IDENTIFICATION OF NEW DIABETES DIAGNOSTIC MARKERS IN SARS-COV-2 INFECTED PATIENTS

(71) Applicants: Firoz Anwar, Jeddah (SA); Turki Mohammed M Alqahtani, Jeddah (SA); Mohammed Abdullah Ali Alghamdi, Jeddah (SA); Mirza R. Baig, Dubai (AE); Fahad A. Al-Abbasi, Jeddah (SA); Ryan Adnan Sheikh, Jeddah (SA); Naif A. R. Almalki, Jeddah (SA); Turky Omar Asar, Alkamil (SA); Azizah Salim Bawadood, Al-Kharj (SA)

(72) Inventors: Firoz Anwar, Jeddah (SA); Turki Mohammed M Alqahtani, Jeddah (SA); Mohammed Abdullah Ali Alghamdi, Jeddah (SA); Mirza R. Baig, Dubai (AE); Fahad A. Al-Abbasi, Jeddah (SA); Ryan Adnan Sheikh, Jeddah (SA); Naif A. R. Almalki, Jeddah (SA); Turky Omar Asar, Alkamil (SA); Azizah Salim Bawadood, Al-Kharj (SA)

(21) Appl. No.: 18/741,561

(22) Filed: Jun. 12, 2024

(51) Int. Cl. G01N 33/573; G01N 33/72; G01N 33/80; G01N 33/92

(52) U.S. Cl. CPC G01N 33/573; G01N 33/721; G01N 33/80; G01N 33/92; G01N 2333/904; G01N 2333/9108; G01N 2333/91188; G01N 2800/042

(57) ABSTRACT

This invention describes a cross-sectional study that examines the trends of long-term complications, particularly the incidence of type 2 diabetes mellitus (T2D), in the Saudi population following SARS-COV-2 infection and vaccination. The study collected blood samples from 203 healthy blood donors and analyzed various parameters, including HbA1c, liver function tests, kidney function tests, and lipid profiles. The results showed a significant increase in HbA1c, urea, creatinine, alkaline phosphatase, lactate dehydrogenase, and triglycerides among diabetic patients compared to non-diabetic individuals. Notably, individuals with blood group O had the highest incidence of T2D. These findings suggest that SARS-COV-2 infection and vaccination are associated with the worsening of T2D symptoms and highlight the need for large-scale studies to understand the long-term metabolic complications of the COVID-19 pandemic, aiming to develop effective prevention and management strategies.

### FIELD OF THE INVENTION

The present disclosure relates to a method for identifying new diabetes diagnostic markers in SARS-COV-2 infected patients, specifically to examine trends of long-term complication post SARS-COV-2 infection and vaccination in diabetes incidence among Saudi population.

### BACKGROUND OF THE INVENTION

In recent times, numerous pieces of evidence have emerged suggesting that individuals experiencing long-term complications post-SARS-COV-2 vaccination may face a diverse range of health issues, including diabetes. While the risks and burdens associated with type 1 diabetes (TID) have been extensively studied and reported, the characterization of type 2 diabetes mellitus (T2D) in this context remains relatively unexplored. To bridge this gap in knowledge, our study aims to investigate the trends in long-term complications post-SARS-COV-2 infection and vaccination regarding diabetes incidence, particularly among the Saudi population.

Over the past five decades, rapid human development and globalization have profoundly altered our environment, human behaviors, and political systems. These changes have contributed significantly to the rise of various metabolic diseases, including diabetes, impacting both developed and developing nations. Diabetes has thus become a global pandemic and a substantial threat to human health, ranking among the top international health concerns. Its economic and social ramifications have been termed as ‘the diabetic apocalypse,’ representing a major health challenge of the 21st century alongside other non-communicable diseases like cardiovascular and chronic kidney diseases.

The economic burden of diabetes is staggering, with global health economic costs projected to reach $1.03 trillion by 2030 and $1.05 trillion by 2045. In Saudi Arabia alone, the economic burden escalated from $2.4 billion in 2015 to $6.5 billion in 2020, with an average annual expenditure of $16,752 per person, solely for diabetes-related expenses.

Recent studies have highlighted associations between the onset of new cases of type 1 diabetes and mRNA COVID-19 vaccines across various age groups. However, the underlying mechanisms and patterns of these associations remain unclear. Notably, an increase in type 1 diabetes incidence has been observed following the pandemic and subsequent vaccination campaigns, while no such observations regarding vaccine association with type 2 diabetes mellitus (T2D) have been reported. This raises questions about whether there is a specific association between vaccines and type 1 diabetes, but not with T2D.

There are reports linking SARS-COV-2 infection to the new onset of diabetes due to viral binding, accumulation, and replication in ACE2 receptors expressed on the islets of the pancreas, potentially leading to pancreatitis.

In the view of the foregoing discussion, it is clearly portrayed that there is a need for an invention that seeks to explore and shed light on any potential post-vaccination onset of T2D, along with investigating possible underlying mechanisms.

### SUMMARY OF THE INVENTION

The present disclosure relates to a method for identifying new diabetes diagnostic markers in SARS-COV-2 infected patients. The present invention aims to conduct a cross-sectional analysis of blood profiles from first-time blood donors. The focus was on HbA1c levels in non-diabetic donors who had not been diagnosed with type 2 diabetes (T2D) prior to the current blood donation. Out of 203 healthy subjects, 104 had elevated HbA1c levels indicating a tendency towards T2D diagnosis, while 99 had normal blood profiles. The study followed the SROBE reporting guidelines and identified significant changes in liver function tests (LFT), kidney function tests (KFT), lipid profile, lactate dehydrogenase (LDH), and susceptibility based on blood group.

The present disclosure seeks to provide a method for identifying new diabetes diagnostic markers in SARS-COV-2 infected patients. The method comprises: collecting blood samples from a group of healthy blood donors in pluralities of tubes; performing various blood profiling for age, sex, weight, history of chronic disease, along with other blood related parameters; determining various blood related parameters by performing various experimental techniques; and performing a statistical analysis for finding out the new diabetes diagnostic markers, using the results from performed blood profiling.

In an embodiment, the healthy blood donors includes, donor with age more than 18 years, and having received all three doses of covid-19 vaccine.

In an embodiment, the blood samples are collected in EDTA and non-EDTA tubes for comprehensive blood profiling.

In an embodiment, determining various blood parameters includes: measuring ABO and Rh blood groups using lorn grouping reagent alternating between two automated blood group analyzer, fitted end to end; measuring hemoglobin levels using a hemocue hb 201 analyzer; and measuring Lactate dehydrogenase (LDH), gamma-glutamyl transferase (GGT), triglycerides, and other parameters using an Atellica CH Analyzer.

In an embodiment, a liver function tests revealed alteration in levels of alkaline phosphate in post covid-19 patients.

In an embodiment, a kidney function test revealed altered levels of urea and Creatinine in post covid-19 patients.

In an embodiment, the lipid profile analysis revealed altered triglyceride levels in post covid-19 patients, and wherein lactate dehydrogenase levels are altered in post covid-19 patients.

In an embodiment, statistical analysis revealed that blood donors having blood groups O+, A, and B+ are susceptible irrespective of their age.

An objective of the present disclosure is to provide a method for identifying new diabetes diagnostic markers in SARS-COV-2 infected patients

Another objective of the present disclosure is to analyze the impact of post-COVID-19 infection on partial Liver Function Test, particularly Alkaline phosphatase levels.

Another objective of the present disclosure is to investigate alterations in Kidney Function Test parameters, specifically urea and creatinine, post-COVID-19 infection.

Another objective of the present disclosure is to examine changes in the lipid profile, particularly Triglyceride levels, in individuals post-COVID-19 infection.

Another objective of the present disclosure is to assess alterations in Lactate dehydrogenase levels post-COVID-19 infection.

Yet, another objective of the present disclosure is to identify the susceptibility to post-COVID-19 complications based on blood group, with a focus on O+, A, and B+ groups, across different age groups.

To further clarify advantages and features of the present disclosure, a more particular description of the invention will be rendered by reference to specific embodiments thereof, which is illustrated in the appended drawings. It is appreciated that these drawings depict only typical embodiments of the invention and are therefore not to be considered limiting of its scope. The invention will be described and explained with additional specificity and detail with the accompanying drawings.

### BRIEF DESCRIPTION OF FIGURES

These and other features, aspects, and advantages of the present disclosure will become better understood when the following detailed description is read with reference to the accompanying drawings in which like characters represent like parts throughout the drawings, wherein:

FIG. 1 illustrates a flow chart of a method for identifying new diabetes diagnostic markers in SARS-COV-2 infected patients in accordance with an embodiment of the present disclosure;

FIG. 2 illustrates a table showing the demographic data of the patients in accordance with an embodiment of the present disclosure;

FIG. 3 illustrates a graphs showing variation in liver profile levels among male and female blood donors in accordance with an embodiment of the present disclosure;

FIG. 4 illustrates a graph showing variation in variation in Lipid profile (Total Cholesterol, HDL and LDL) levels in mmol/L among male and female newly diagnosed by T2D in accordance with an embodiment of the present disclosure; and

FIG. 5 illustrates a table showing variation in significant values of various blood parameters among newly diagnosed by T2D and Non diabetic patients in accordance with an embodiment of the present disclosure.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

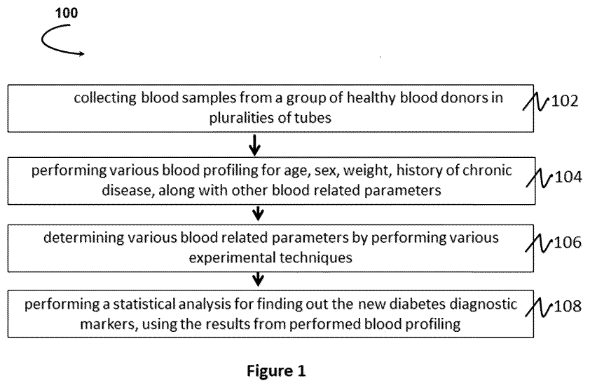

FIG. 1 illustrates a flow chart of a method (100) for identifying new diabetes diagnostic markers in SARS-COV-2 infected patients in accordance with an embodiment of the present disclosure.

Referring to FIG. 1, the method (100) includes pluralities of steps as described below,

At step (102), the method (100) includes collecting blood samples from a group of healthy blood donors in pluralities of tubes.

At step (104), the method (100) includes performing various blood profiling for age, sex, weight, history of chronic disease, along with other blood related parameters.

At step (106), the method (100) includes determining various blood related parameters by performing various experimental techniques.

At step (108) the method (100) includes performing a statistical analysis for finding out the new diabetes diagnostic markers, using the results from performed blood profiling.

In an embodiment, the healthy blood donors includes, donor with age more than 18 years, and having received all three doses of covid-19 vaccine.

In an embodiment, the blood samples are collected in EDTA and non-EDTA tubes for comprehensive blood profiling.

In an embodiment, determining various blood parameters includes: measuring ABO and Rh blood groups using lorn grouping reagent alternating between two automated blood group analyzer, fitted end to end; measuring hemoglobin levels using a hemocue hb 201 analyzer; and measuring Lactate dehydrogenase (LDH), gamma-glutamyl transferase (GGT), triglycerides, and other parameters using an Atellica CH Analyzer.

In an embodiment, a liver function tests revealed alteration in levels of alkaline phosphate in post covid-19 patients.

In an embodiment, a kidney function test revealed altered levels of urea and Creatinine in post covid-19 patients.

In an embodiment, the lipid profile analysis revealed altered triglyceride levels in post covid-19 patients, and wherein lactate dehydrogenase levels are altered in post covid-19 patients. In an embodiment, statistical analysis revealed that blood donors having blood groups O+, A, and B+ are susceptible irrespective of their age.

The present invention relates to examine the trends of long-term complications, including the incidence of type 2 diabetes mellitus (T2D), in the Saudi population following SARS-COV-2 infection and vaccination.

A total of 500 healthy donors who had received the COVID-19 vaccine and were previously exposed to the SARS-COV-2 virus were selected for this study. Donors without prior SARS-COV-2 infection were excluded. Ethical clearance was obtained from the Research Unit of Biomedical Ethics at King Abdulaziz University under approval reference 16-23, dated 15-1-2023, issued by the Chairman of the Research Ethics Committee. The study strictly followed protocols recommended by health authorities.

Blood samples were collected from the donors using EDTA and non-EDTA tubes to facilitate various necessary blood profiling, including age, sex, weight, history of chronic diseases, and other parameters as detailed in the results section.

The inclusion criteria for this invention encompassed individuals who had received all three doses of the COVID-19 vaccine and were aged 18 years and above. Conversely, the exclusion criteria included individuals who had not been exposed to any chronic infection following vaccination, those with metabolic diseases such as diabetes, hypertension, stress, gout, pregnant and lactating women, and anyone with lower hemoglobin levels according to the Ministry of Health in Saudi Arabia.

Regarding the sample size, the original intention was to include a large sample of over 300 donors. However, due to limitations in the number of available donors, the final sample size was reduced to 204 individuals.

The experimental techniques employed in this invention involved the determination of ABO/Rh blood groups using Lorn grouping reagent from Lorn Laboratory Ltd, UK. This process was carried out using two automated blood group analyzers connected end to end to minimize human errors. Hemoglobin levels were measured using the HemoCue Hb 201 device, while HbA1c levels were assessed using the Bio-Rad variant II Turbo system. Other parameters such as Lactate Dehydrogenase L-P (LDLP), Gamma-Glutamyl Transferase (GGT), Triglycerides, and additional parameters were analyzed using the Atellica CH Analyzer from Siemens Healthineers in Erlangen, Germany.

For statistical analysis, the Statistical Package for Social Science (SPSS) version 26 from Chicago, IL, was employed. Independent t-tests with post hoc analysis were conducted, with statistical significance set at p-values less than 0.05 or 0.001, indicating meaningful findings.

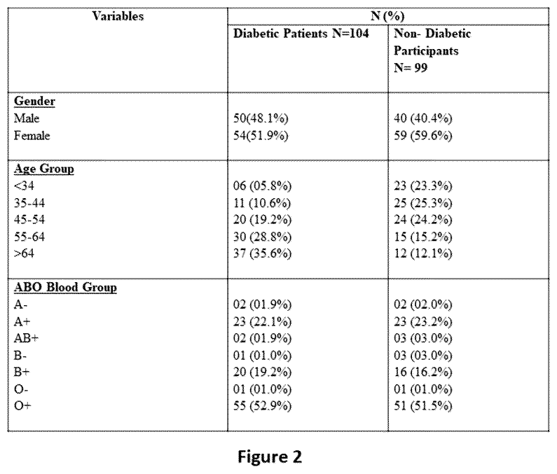

FIG. 2 illustrates a table showing the demographic data of the patients in accordance with an embodiment of the present disclosure.

Referring to table shown in FIG. 2, an analysis is conducted involving diabetic individuals (N=104) comprising 54 males and 50 females, as well as non-diabetic individuals (N=99) comprising 40 males and 59 females, all of whom had received different COVID-19 vaccines. These individuals were categorized based on their ABO blood group, along with various demographic characteristics including age, sex, nationality, Rh factors, and different blood group types, during May 2023.

The plurality of observations revealed a consistent increase in the number of donors as the age group increased, with the highest number of diabetic donors falling in the age group above 64 years and the lowest number in the age group under 34 years. Conversely, for non-diabetic donors, the pattern was nearly reversed. Interestingly, we did not observe any significant differences in the distribution of ABO blood groups between diabetic and non-diabetic individuals.

Among the diabetic donors, the highest number of newly diagnosed patients belonged to the O+ blood group, while the lowest were from the O− and B− blood groups. This data is summarized in the table provided in FIG. 2.

A notable difference in HbA1c levels was observed between male and female donors. Males exhibited higher HbA1c percentages, with an average of 8.24, compared to females who averaged 7.61, regardless of age, blood group, or other demographic factors. Females tended to have higher but well-controlled HbA1c levels compared to males, who displayed uncontrolled HbA1c values. Additionally, a significant variation with a p-value greater than 0.01 was noted in HbA1c percentages among new donors. The HbA1c levels were recorded at an average of 7.90±2.28 for males and 5.88±5.50 for females, irrespective of demographic factors such as age, blood group, or other demographics.

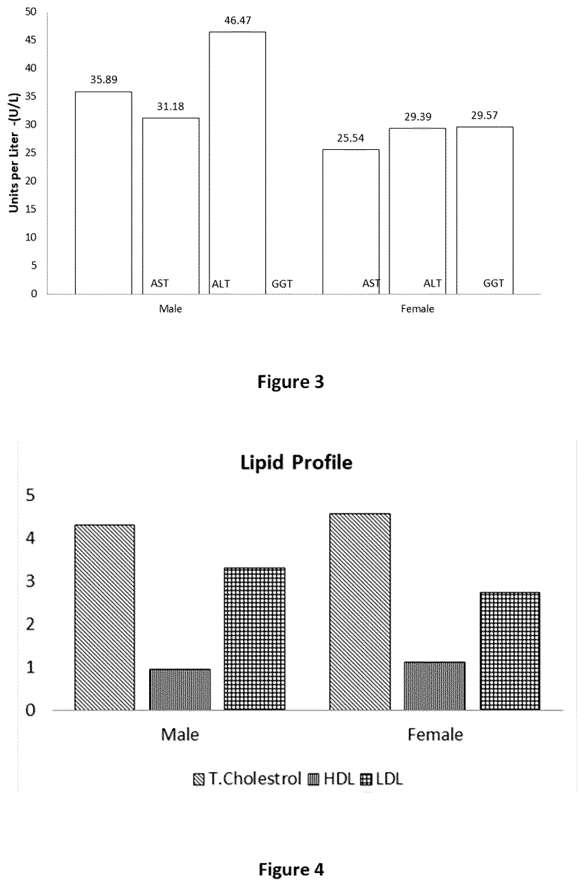

FIG. 3 illustrates a graphs showing variation in liver profile levels among male and female blood donors in accordance with an embodiment of the present disclosure.

Referring to FIG. 3, there was no significant variation in ALT (35.89), (25.54) and AST (31.18), (29.39) of both male and female with abnormal HbA1c value. However, GGT was reported at 46.47 with a significant higher level compared to female at 29.57 among male and female donors respectively. Alkaline phosphatase physiologically dephosphorylates many compound, and major component liver of liver metabolism.

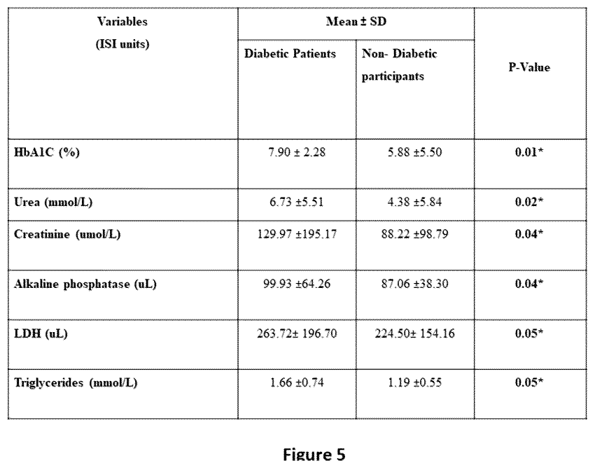

FIG. 4 illustrates a graph showing variation in variation in Lipid profile (Total Cholesterol, HDL and LDL) levels in mmol/L among male and female newly diagnosed by T2D in accordance with an embodiment of the present disclosure.

Referring to FIG. 4, both male and female donors with newly diagnosed elevated level of HbA1c exhibited the same pattern of Total cholesterol, HDL and LDL with no significant difference both in diabetic and non-diabetic patients.

FIG. 5 illustrates a table showing variation in significant values of various blood parameters among newly diagnosed by T2D and Non diabetic patients in accordance with an embodiment of the present disclosure.

Referring to table shown in FIG. 5, below described observations have been revealed.

Alkaline phosphatase, responsible for dephosphorylating various compounds and a crucial component of liver metabolism, showed a significant variation (p>0.04) between two population subsets newly diagnosed with T2D, with values of 99.93±64.26 and 87.06±38.30, respectively.

Triglyceride levels exhibited a significant increase (p>0.05) in 50% of donors, measuring at 1.66±0.74 mmol/L compared to the rest of the donors at 1.19±0.55 mmol/L.

Urea levels also displayed significant variation (p>0.02), recording values of 6.73±5.51 mmol/L and 4.38±5.84 mmol/L, respectively. Similarly, there was a significant difference (p>0.04) in Creatinine levels, with values of 129.97±195.17 umol/L and 88.22±98.79 umol/L observed between the two groups. These variations were consistent across demographics.

Lactate dehydrogenase (LDH), an enzyme present in various tissues, showed a significant elevation (p>0.05) in 50% of the population, measuring at 263.72±196.70 uL compared to other donors at 224.50±154.16 uL. LDH levels can be elevated in conditions like liver disease, heart attacks, and infectious diseases such as HIV, meningitis, and viral infections.

In an embodiment, the serum profile showed normal values for electrolytes (Sodium, Potassium, Chloride, Calcium, Magnesium, Phosphate), Total protein, Albumin, Uric acid, AST, ALT, GGT, TBIL, Cholesterol, HDL, LDL, Hemoglobin (Hb), Neutrophils (Neu), Lymphocytes, and the Neu/Lymph ratio. Although there were observed changes in these parameters, they were not significantly altered and could not be linked to any significant changes.

The present invention explored the trends of long-term complications, particularly the incidence of type 2 diabetes mellitus (T2D), in the Saudi population following SARS-COV-2 infection and vaccination. The key findings of this invention reveal several important insights.

Contrary to global trends, the invention observed a slightly higher but non-significant increase in diabetes among females compared to males after the COVID-19 pandemic. This finding challenges the prevailing notion that diabetes disproportionately affects the male population. Furthermore, the data showed the highest incidence of T2D in the age group over 64 years, countering the assumption that increasing age and male sex are independently associated with new cases of diabetes.

Interestingly, the invention identified blood group O as having the highest incidence of T2D (53%), followed by A (25%), B (20.2%), and AB (1.9%). These results suggest a possible role of ABO blood group in the susceptibility to T2D development following SARS-COV-2 infection and vaccination. This finding highlights the importance of considering the influence of genetic factors in the metabolic response to the COVID-19 pandemic.

The invention also revealed significant alterations in various blood parameters among diabetic and non-diabetic patients. Notably, the researchers observed substantial changes in HbA1c, urea, creatinine, alkaline phosphatase, lactate dehydrogenase (LDH), and triglycerides. The elevated levels of LDH and triglycerides in diabetic patients suggest their potential as biomarkers for the early detection of diabetes in the post-vaccination era.

In conclusion, the present invention provides compelling evidence that SARS-COV-2 infection and vaccination are associated with the worsening of T2D symptoms, as indicated by the significant increase in HbA1c levels and the observed alterations in liver, kidney, and lipid profiles. These findings underscore the critical need for large-scale population-based studies to better understand the long-term metabolic complications, including the incidence of T2D, following the COVID-19 pandemic. This knowledge is crucial for developing effective prevention and management strategies, especially for high-risk populations, to mitigate the burden of diabetes in the region.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A blood profile analysis method for identification of new diabetes diagnostic markers, comprising:
collecting blood samples from a group of healthy blood donors in pluralities of tubes;
performing various blood profiling for age, sex, weight, history of chronic disease, along with other blood related parameters;
determining various blood related parameters by performing various experimental techniques; and
performing a statistical analysis for finding out the new diabetes diagnostic markers, using the results from performed blood profiling.

2. The method of claim 1, wherein the healthy blood donors includes, donor with age more than 18 years, and having received all three doses of covid-19 vaccine.

3. The method of claim 1, wherein the blood samples are collected in EDTA and non-EDTA tubes for comprehensive blood profiling.

4. The method of claim 1, wherein determining various blood parameters includes:
measuring ABO and Rh blood groups using lorn grouping reagent alternating between two automated blood group analyzer, fitted end to end;
measuring hemoglobin levels using a hemocue hb 201 analyzer; and
measuring Lactate dehydrogenase (LDH), gamma-glutamyl transferase (GGT), triglycerides, and other parameters using an Atellica CH Analyzer.

5. The method of claim 4, wherein a liver function tests revealed alteration in levels of alkaline phosphate in post covid-19 patients.

6. The method of claim 4, wherein a kidney function test revealed altered levels of urea and Creatinine in post covid-19 patients.

7. The method of claim 4, wherein the lipid profile analysis revealed altered triglyceride levels in post covid-19 patients, and wherein lactate dehydrogenase levels are altered in post covid-19 patients.

8. The method of claim 1, wherein statistical analysis revealed that blood donors having blood groups O+, A, and B+ are susceptible irrespective of their age.
